(19) United States
(12) Patent Application Publication
(10) Pub. No.: US 2012/0251385 A1
(43) Pub. Date: Oct. 4, 2012

(54) STERILIZATION METHOD OF A FOOD COOLING DEVICE

(76) Inventor: Chin-Chan Hsieh, Nantou City (TW)

(21) Appl. No.: 13/075,222

(22) Filed: Mar. 30, 2011

(51) Int. Cl. A61L 2/07

(52) U.S. Cl. 422/26

(57) ABSTRACT

A sterilization method of a food cooling device employing a temperature difference effect, includes a sterilizing space to accommodate energy conductive tubes with cold water within and a steam transited to the sterilizing space for a fast temperature difference phenomenon and further a temperature difference effect created by low-temperature fluid inside and steam outside the energy conductive tubes in which the temperature difference effect strengthens lethality to colon bacillus or Salmonella and separates residual food from the energy conductive tubes' walls for cleaning effectively delivered by the energy conductive tubes and food healthily processed by the food cooling device.

### BACKGROUND OF THE INVENTION

1. Field of the Invention

The present invention relates to a sterilization method of a food cooling device, especially to a sterilization method with a food cooling device's energy conductive tubes bringing a fast temperature difference by which the effect of cleaning and sterilization is delivered.

2. Description of the Prior Art

As a result of diversified marketing channels substantially contributing to sales volumes of various traditional delicacies as well as more and more foods supplied to increased fastfood junkies by food manufacturers in recent years, the conventional food processing has been transferred to automatic production from manual operation for increased output and curtailed manpower and throughput time. Conventionally, some completely heated food materials such as sauce, curry, braised pork, filling, boiled rice, and flour dough are directly rested for cooling which might be quite slow and unhealthy with dust, fly, or mosquito contacting and result in potential burn in case of these materials hereinbefore improperly arranged or dropped;

To save time for cooling food materials, the inventor has developed a brand-new food cooling device (Taiwan (R.O.C.) Patent No. 1330249) with a driving mechanism (for a reciprocating & positioning motion) on which there is a heat exchanger comprising a plurality of vacuum-encapsulated stainless steel energy conductive tubes that follows the driving mechanism to move and are designed to accommodate low-temperature fluid within and reach a container full of high-temperature food for food's thermal energy (or temperature) inside the container quickly removed (or reduced) according to the heat exchange theory, less energy resource wasted, and a device user's fewer cost.

Despite its design being popular with users in the industry, the food cooling device hereinbefore featuring energy conductive tubes inserted into food for cooling is difficultly cleaned owing to plenty of tubes arranged to be a matrix in restricted spacing and no proper sterilizer available in the market for health food and energy conductive tubes properly sterilized before usage.

### SUMMARY OF THE INVENTION

The objective of the present invention is to provide a sterilization method of a food cooling device by which food can be sterilized fast, effectively and healthily.

The sterilization method can be applied to a food cooling device directly contacting with food materials wherein the food cooling device comprises at least plenty of energy conductive tubes and a sterilizing space for the energy conductive tubes selectively inserted and the sterilization method of the food cooling device delivering the said invention comprises:

A sterilizing space for the energy conductive tubes inserted; a steam introduced into the sterilizing space for a fast temperature difference phenomenon developed on the energy conductive tubes' walls and further a temperature difference effect by which the purpose of sterilization and food separated is delivered.

### BRIEF DESCRIPTION OF THE DRAWINGS

The drawings disclose an illustrative embodiment of the present invention which serves to exemplify the various advantages and objects hereof, and are as follows:

FIG. 1 is the perspective view of the present invention of a food cooling device for sterilization;

FIG. 2 and FIG. 3 are the schematic diagrams for operating the sterilization method of a food cooling device in the present invention;

FIG. 4 is the perspective view of the present invention in the second embodiment;

FIG. 5 is the side view of the present invention in the second embodiment;

FIG. 6 is the schematic diagram for operating the auxiliary cooling device.

### DETAILED DESCRIPTION OF THE PREFERRED EMBODIMENT

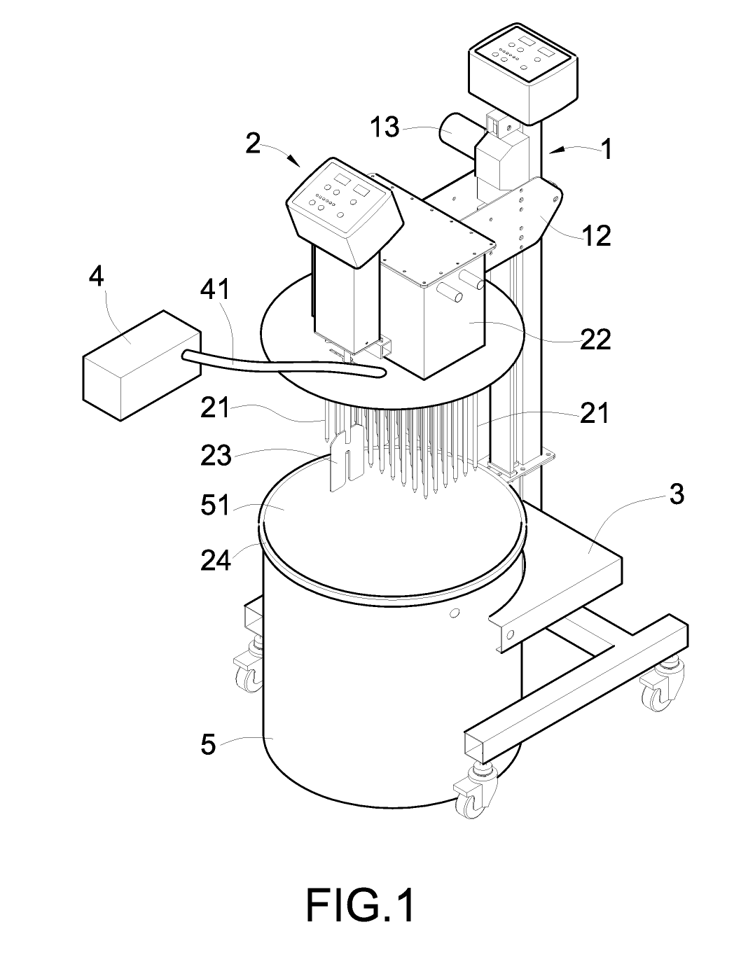

Referring to FIG. 1 which indicates the present invention of a food cooling device comprising a driving mechanism 1, a heat exchanger 2, a steam generator 4, and a container 5;

The driving mechanism 1 is intended for a reciprocating and positioning motion with a lever arm 12 driven by a motor 13;

The heat exchanger 2 comprising a plurality of energy conductive tubes 21 is installed on the lever arm 12 and moved by the activated lever arm 12 and the vacuum-encapsulated metal energy conductive tubes 21 with closed cavities for working fluid constantly cycling between liquid and gaseous states as well as convecting between gas (releasing heat at a heat release portion) and liquid (absorbing heat at a heat absorption portion) deliver both constant temperature rapidly achieved on the cavities' surfaces and heat transfer because of thermal energy quickly transferred to a heat release portion for temperature inside a container 5 rapidly and uniformly reduced according to phase change with a heat absorption portion of the energy conductive tubes 21 inserted into the high-temperature container 5; additionally, a silicon gasket 24 is further installed under a connecting unit 22 in order to strengthen airtight joint between the connecting unit 22 and the container 5 and lift both pressure inside a sterilizing space 51 as well as sterilizing effect;

With food cooled down and taken out, the energy conductive tubes 21 are placed into the sterilizing space 51 inside the container 5 for a steam introduced to create a fast temperature difference out of the steam on walls of the energy conductive tubes 21 and further a temperature difference effect which is used to deliver sterilization and food separated.

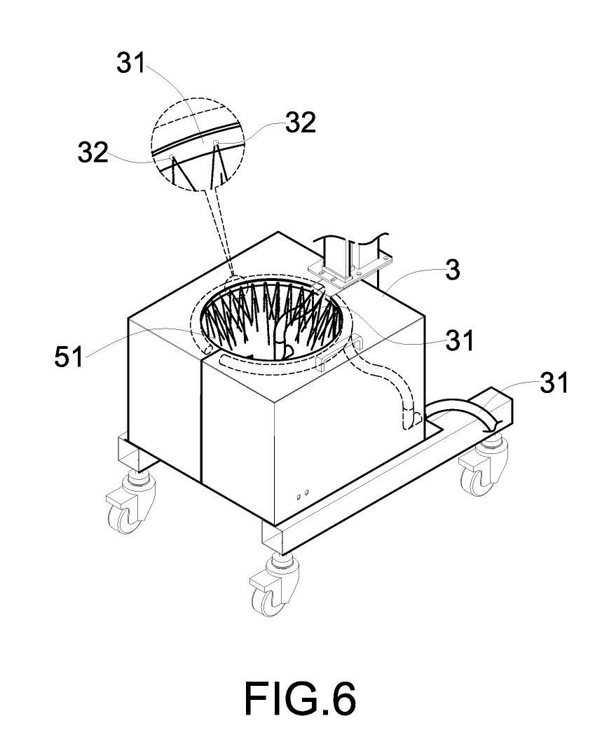

For food to be cooled by a user who inserts the energy conductive tubes 21 into high-temperature food held by the container 5, the fluid with a low boiling point is repeatedly boiling (with heat absorbed) and condensing (with heat released) inside the energy conductive tubes 21 because of phase change to rapidly remove food's thermal energy and cool food within the container 5; furthermore, water flows supplied to an auxiliary cooling device 3 from an aqueduct 31 are sprayed around the container 5 via outlets 32 (FIG. 6) and contribute to cooling by means of inner and outer heat exchange to deliver dual temperature drop for temperature of food within the container 5 rapidly reduced and waiting cooling time saved in the present invention;

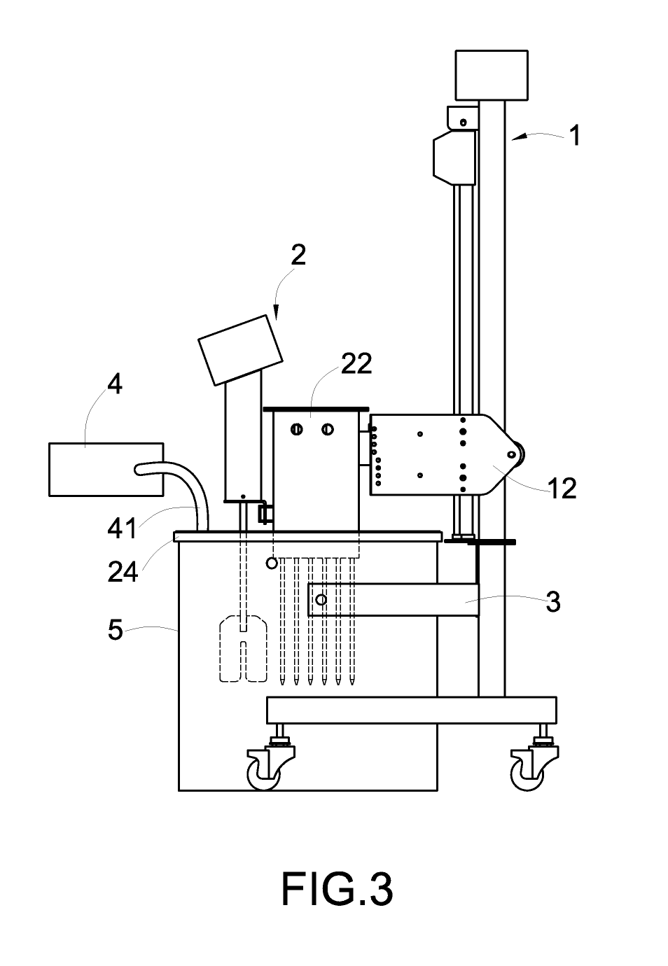

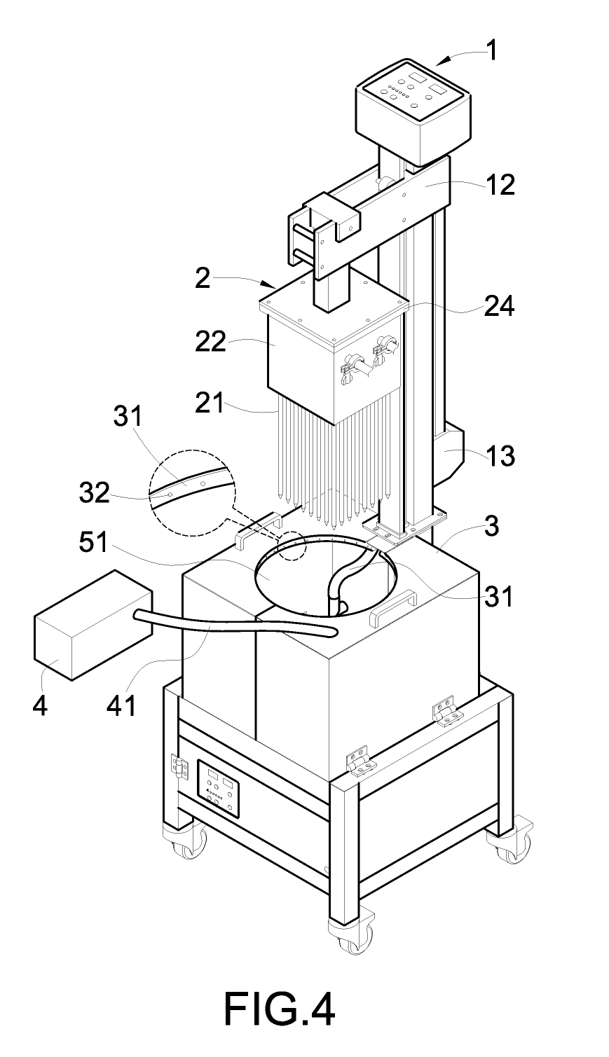

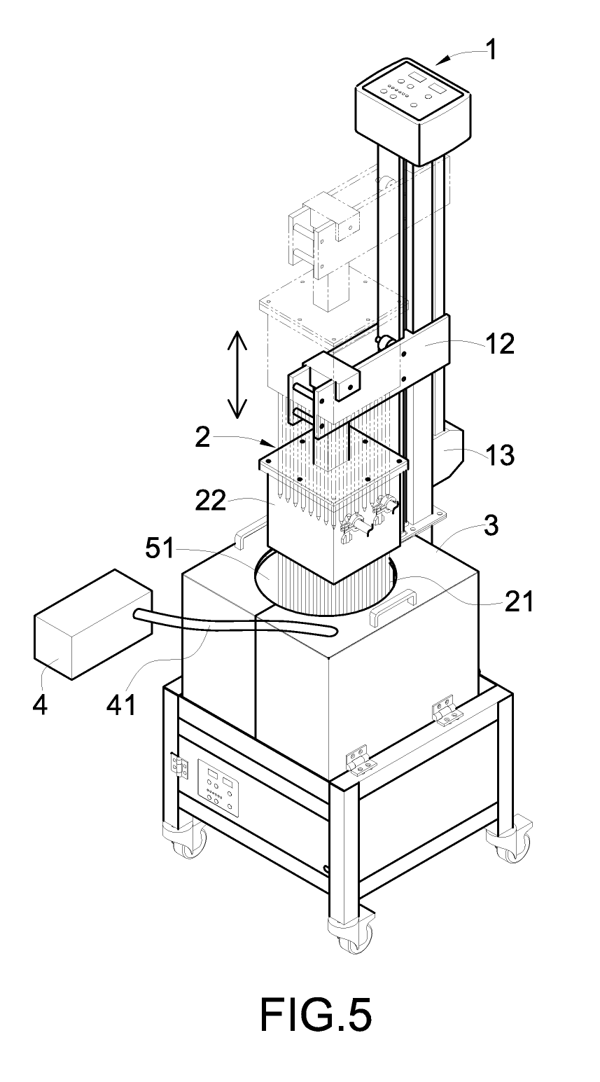

As shown in FIGS. 3 and 5 for practical operations, the energy conductive tubes 21 of the heat exchanger 2 waiting to be cleaned and sterilized with food completely cooled should be placed into the sterilizing space 51 of the container 5 by moving the lever arm 12 of the driving mechanism 1 and the steam sterilization in the present invention is interpreted as followed:

Make use of siphonage to release low-temperature fluid, which is contained in the connecting unit 22 and contacts with a heat release portion of the energy conductive tubes 21, for the energy conductive tubes 21 kept at the first temperature;

Place a heat absorption portion of the energy conductive tubes 21 into the sterilizing space 51 of the container 5;

Transit steam into the sterilizing space 51 via a steam pipe 41 and keep the energy conductive tubes 21 at the higher second temperature for a fast temperature difference phenomenon and further a temperature difference effect which is used to deliver sterilization and food separated.

For the energy conductive tubes 21 heated to the second temperature from the first one, the temperature difference should be greater than 100 degrees Celsius for the temperature difference effect kept during the steam transited into the sterilizing space 51. As low-pressure steam sterilization, the sterilization available to the energy conductive tubes 21 in the present invention is performed under a condition of saturated steam between 115 and 126 degrees Celsius for 3˜15 minutes.

The present invention purposely relies on a temperature difference effect to make food adhered on the energy conductive tubes 21 and protein (or protein enzyme) of bacteria on food coagulated and denatured at a low-pressure saturated steam for a sterilization effect; the present invention as an effective, safe and clean method to handle food on the energy conductive tubes 21 heats and sterilizes the energy conductive tubes 21 in a way of a temperature difference effect being attributed to a high temperature difference for microbes eradicated. In addition, the said energy conductive tubes 21 with their walls also affected by a temperature difference effect contribute to not only adherent food separated from the energy conductive tubes 21 but also exclusion of inconvenience in cleaning the energy conductive tubes 21.

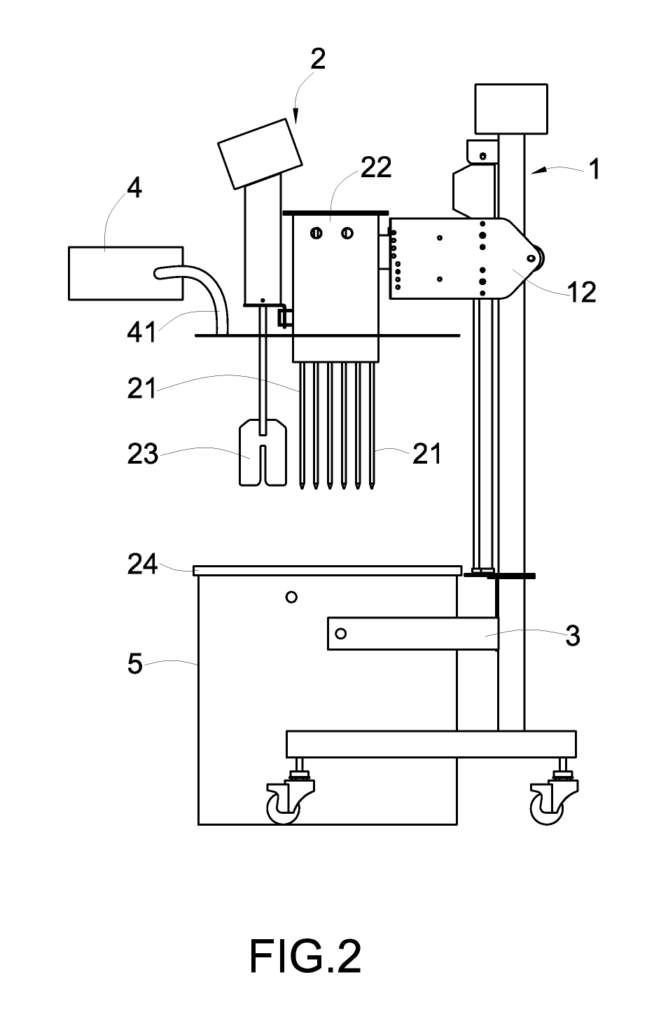

As shown in FIGS. 1 and 2, an agitating device 23 ingeniously installed at the front of the connecting unit 22 by the inventor is effective in constantly rotating and agitating sticky food (such as curry sauce and meat paste unfavorable to cooling at a uniform temperature) inside the container 5 and allows food to be endlessly moved and uniformly contact with the energy conductive tubes 21 to advantageously lower the container's temperature. To deliver the said steam and temperature difference effect, the agitating device 23 is first rinsed with hot water followed by cleaning of temperature difference in food.

Many changes and modifications in the above described embodiment of the invention can, of course, be carried out without departing from the scope thereof. Accordingly, to promote the progress in science and the useful arts, the invention is disclosed and is intportioned to be limited only by the scope of the appportioned claims.

What is claimed is:

1. A sterilization method of a food cooling device applied to a food cooling equipment directly contacting with food for heat emission wherein the food cooling device comprises a plurality of energy conductive tubes and a sterilizing space for steam pipes selectively inserted and the sterilization method comprises:
providing a sterilizing space for the energy conductive tubes accommodated in a container, a steam transited into the sterilizing space to create a fast temperature difference phenomenon out of the steam on walls of the energy conductive tubes and further a temperature difference effect which is used to deliver sterilizing effect and food separated effect.

2. The sterilization method of the food cooling device according to claim 1 wherein the energy conductive tubes are installed on a heat exchanger which allows the energy conductive tubes to be inserted into the container's sterilizing space via a driving mechanism's moving lever arm.

3. The sterilization method of the food cooling device according to claim 1 wherein the steam transited to the sterilizing space from one steam pipe keeps the energy conductive tubes at a higher first temperature to create the fast temperature difference phenomenon and further the temperature difference effect which is used to deliver sterilizing effect and food separated effect.

4. The sterilization method of the food cooling device according to claim 3 wherein the energy conductive tubes are heated to the first temperature from a second temperature reaching a temperature difference greater than 100 degree Celsius.

5. The sterilization method of the food cooling device according to claim 3 wherein sterilization of the energy conductive tubes is performed under a condition of saturated steam between 115 and 126 degrees Celsius for 3˜15 minutes.
